(19) United States
(12) Patent Application Publication
(10) Pub. No.: US 2025/0213349 A1
(43) Pub. Date: Jul. 3, 2025

(54) DEVICE AND METHOD FOR APPLICATION OF BIOINDUCTIVE IMPLANT PATCH IN ARTHROSCOPIC SURGERY

(71) Applicant: JJJ MEDICAL LLC, MARIETTA, GA (US)

(72) Inventors: JEREMY SMITH, CHARLESTON, SC (US); JUSTIN SMITH, FORT MILL, SC (US); JEFFREY BROADRICK, ATHENS, GA (US); JAMIE WAYNE LINN, ELK RIVER, MN (US)

(21) Appl. No.: 18/741,971

(22) Filed: Jun. 13, 2024

Related U.S. Application Data
(60) Provisional application No. 63/617,243, filed on Jan. 3, 2024.

(51) Int. Cl. A61F 2/08; A61F 2/00

(52) U.S. Cl. CPC A61F 2/0811; A61F 2/0805; A61F 2002/0072

(57) ABSTRACT

A novel device for bioinductive implant placement in an in vivo surgical site, and method of use thereof, is disclosed. Sequential actuation of a series of levers (or other actuation means) operates to allow for expansion or splaying of a bioinductive implant patch, such as a patch comprised of collagen fibers, over an in vivo surgical site such as a torn tendon in a shoulder rotator cuff. With the patch expanded, it may be surgically fixed in place and provide for more efficient and thorough healing and repair of the tendon.

### CROSS-REFERENCE TO RELATED APPLICATIONS

This claims priority under 35 U.S.C. § 119 (e) to U.S. provisional application entitled “DEVICE AND METHOD FOR APPLICATION OF BIOINDUCTIVE IMPLANT PATCH IN ARTHROSCOPIC SURGERY,” filed on Jan. 3, 2024 and assigned application Ser. No. 63/617,243, the entire contents of which are hereby incorporated by reference.

### BACKGROUND

The present invention relates to devices and methods for surgery and, more particularly, to a novel and improved surgical device that enables in vivo application of a bioinductive implant patch in an arthroscopic surgical environment.

Surgical repair of torn tendons is commonly performed arthroscopically. One common arthroscopic surgery is for the repair of a torn rotator cuff in a shoulder. Depending on the severity and nature of the tear, surgeons may elect to employ any number of surgical methods to repair a torn rotator cuff. A traditional approach involves threading sutures through the tendon so that the tendon can be put back into place by pulling the sutures taut. The sutures are then held taut by an anchor set into the tubercle of the humerus bone. The tendon may then heal in place, reattaching itself to the humerus.

Recent advancements in tendon repair, particularly in rotator cuff repair, have shown that the placement of a bioinductive implant patch comprised of collagen over the torn tendon will promote faster and more thorough healing of the tendon tear. Consequently, it is becoming more common for surgeons to place a bioinductive implant over the tear after the tendon has been sutured and anchored as described above. For less severe tears, surgeons may elect to forego using sutures and anchors in favor of using only a bioinductive implant.

Therefore, there is a need in the art for an improved device and method for placement of a bioinductive implant during arthroscopic surgery.

### SUMMARY

A novel device for bioinductive implant placement in an in vivo surgical site, and method of use thereof, is disclosed. Sequential actuation of a series of levers (or other actuation means) operates to allow for expansion or splaying of a bioinductive implant patch, such as a patch comprised of collagen fibers, over an in vivo surgical site such as a torn tendon in a shoulder rotator cuff. With the patch expanded, it may be surgically fixed in place and provide for more efficient and thorough healing and repair of the tendon.

An exemplary embodiment of a bioinductive implant placement device according to the solution includes an ergonomic housing containing a plurality of manual actuators. Each actuator extends external to the housing for actuation by a user as the user holds the housing. A plurality of concentrically and slidably arranged, or concentrically nested, components extend from the housing to define a surgery application end that is distal to the housing. The plurality of concentrically arranged components comprise a main shaft slidably nested within a bottom tab assembly slidably nested within a tubular sheath. Notably, the main shaft is a stationary, fixed datum relative to which the bottom tab assembly and tubular sheath retract or extend. An application head of a patch-splayer component is coupled to the main shaft such that when a pulling or tensioning force (via one of the levers, for example) is applied to the patch-splayer component, the application head of the patch-splayer component deforms by collapsing from its default expanded state. The application head may be operable to deform and transition between its expanded state and collapsed state by virtue of being shape-set and/or being constructed from a flexible, i.e. elastically deformable, material such as, for example, Nitinol. The plurality of manual actuators are each uniquely associated with one of the patch-splayer component, bottom tab assembly, and tubular sheath. As such, sequential actuation of the plurality of manual actuators causes transitions of the patch-splayer component, bottom tab assembly, and tubular sheath so that the surgical application end transitions between a loaded state and a deployed state. When in the loaded state, the surgical application end is operable to fold the bioinductive implant patch for insertion into an in vivo surgical site, and when in the deployed state the surgical application end is operable to splay and release the bioinductive implant patch onto the in vivo surgical site.

The patch-splayer component and/or its application head may be comprised of Nitinol in a preferred embodiment. The patch-splayer component and/or its application head may be comprised of a wire or a laser-cut tube. The tubular sheath may define a slot configured for accommodating the patch when in the loaded state. The bottom tab assembly may define a tab that extends beneath the bioinductive implant patch when the surgical application end is in the deployed state, to support the patch and ease proper positioning of the patch within the in vivo surgical site. When the application end is in the loaded state, the bioinductive implant patch is folded around the main shaft and a tensioning force is applied to the patch splayer component such that its application head is positioned within the walls of the tubular sheath. When expanded, the application head of the patch-splayer component is free to take a default shape (generally, circular or oval in a preferred embodiment) that operates to unfold and splay the bioinductive implant patch from around the main shaft.

An exemplary method for using a bioinductive implant placement device according to the solution comprises loading the bioinductive implant device into a loaded state by placing a bioinductive implant patch onto the main shaft and retracting the application head into the tubular sheath such that the bioinductive implant patch is folded around the main shaft, held in place by the bottom tab assembly, and positioned within the slot defined at the distal end of the tubular sheath. Next, the bioinductive implant device is inserted into an in vivo surgical site. A first manual actuator of the plurality of manual actuators is actuated to retract the tubular sheath relative to the other concentrically and slidably nested components. A second manual actuator of the plurality of manual actuators is actuated to allow the application head of the patch-splayer component to expand to a default shape and splay the bioinductive implant patch over the in vivo surgical site. At that point, the bioinductive implant patch may be surgically fixed in place in the in vivo surgical site. A third manual actuator of the plurality of manual actuators is actuated to retract the bottom tab assembly from beneath the bioinductive implant patch. Finally, with the bioinductive implant patch fixed in place, the surgical end of the device may be removed from the in vivo surgical site. Optionally, prior to removing the surgical end from the in vivo surgical site, a user may elect to retract the application head back into the tubular sheath (or, depending on perspective, extend the tubular sheath such that the flexible and/or shape-set application head collapses).

### BRIEF DESCRIPTION OF THE DRAWINGS

In the Figures, like reference numerals refer to like parts throughout the various views unless otherwise indicated. For reference numerals with letter character designations such as “215A” or “215B,” the letter character designations may differentiate two like parts or elements present in the same Figure. Letter character designations for reference numerals may be omitted when it is intended that a reference numeral to encompass all parts having the same reference numeral in all Figures.

FIGS. 1A-1B illustrate use of a bioinductive implant placement device according to the solution in an arthroscopic surgical application

FIG. 2A is an exploded view of an exemplary embodiment of a bioinductive implant placement device according to the solution;

FIG. 2B illustrates an exemplary and alternative patch-splayer from that which is illustrated in FIG. 2A, the exemplary and alternative patch-splayer comprised of a laser cut and shape-set Nitinol tube welded to a stainless tube and configured to slidably receive a main shaft;

FIG. 3 is a perspective view and cutaway view of the exemplary embodiment of a bioinductive implant placement device shown in FIG. 2;

FIG. 4A illustrates the exemplary embodiment of the solution for a bioinductive implant placement device in a loaded state, ready for insertion to an in vivo surgical site;

FIG. 4B illustrates the exemplary embodiment of the solution for a bioinductive implant placement device in a deployed state, with the shape-set patch-splayer expanded and ready for release from the bioinductive implant patch;

FIGS. 5A-5D illustrate the actuation sequence of levers of a bioinductive implant placement device according to the solution for application and release of a bioinductive implant patch to a surgical site, such as a torn tendon;

FIGS. 6A-6C illustrate in more detail the use of the bioinductive implant placement device according to the solution, with FIG. 6A illustrating arthroscopic access to an in vivo surgical site for an exemplary tendon repair in a shoulder, FIG. 6B illustrating an application end of a bioinductive implant placement device according to the solution in a loaded state and positioned over the in vivo surgical site, and FIG. 6C illustrating the application end of the bioinductive implant placement device according to the solution in a deployed state to place a bioinductive patch over and onto the in vivo surgical site.

### DETAILED DESCRIPTION

Various embodiments, aspects and features of the present invention encompass a surgical device for application and placement of a bioinductive implant, such as a collagen-based patch, during arthroscopic surgery. Further configurations and advantages and uses of the solution will occur to those of skill in the art reviewing the figures and description that follows.

In this description, the term bioinductive implant means any device operable to promote healing of tendons through inducement of new tissue growth. A typical bioinductive implant may be in the form of a “patch” or relatively thin pad. As one of ordinary skill in the art would acknowledge and understand, bioinductive implants may contain collagen and are commonly used to treat patients with deteriorated tendons due to rotator cuff disease and/or patients with rotator cuff tendon tears.

In this description, “in vivo” means performed or taking place in a living organism. As such, use of the term “in vivo application site” or “in vivo surgical site” or the like would be understood by one of ordinary skill in the art to refer to the surgical location or site within a living organism's body (such as, but not limited to, a human) such as, for example, a torn tendon of a rotator cuff.

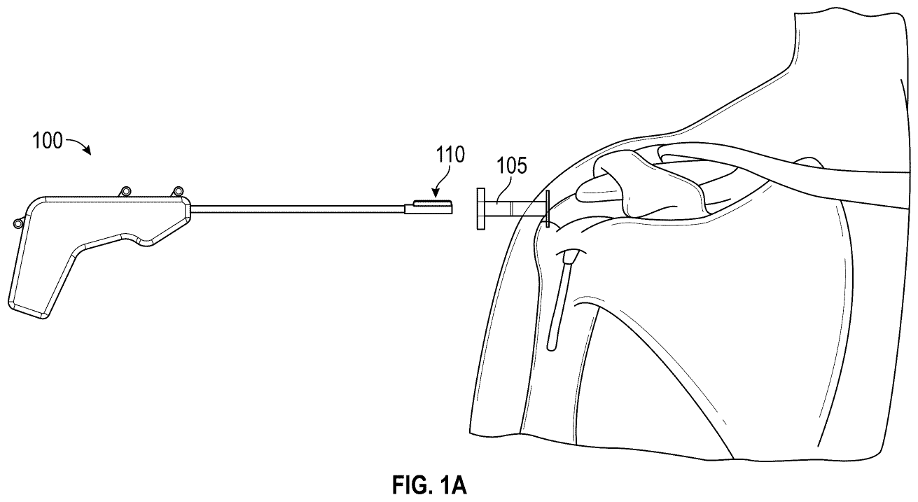

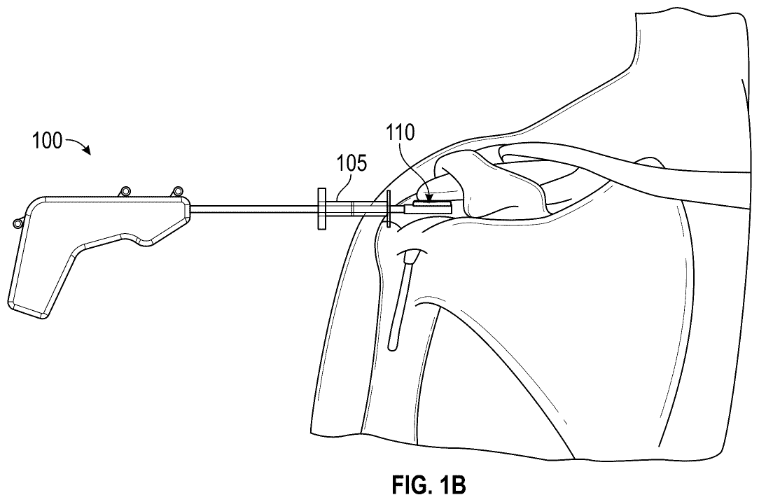

Referring to FIGS. 1A-1B, illustrated is the use of a bioinductive implant placement device 100 according to the solution in an arthroscopic surgical application. As can be understood from the illustrations, the bioinductive implant placement device 100 is being employed in an arthroscopic surgery for tendon repair in a shoulder. An insertion point into the shoulder for access to the in vivo surgical site has been created and is “held open” by a tube 105. A bioinductive implant placement device 100 in a loaded state (FIG. 1A) is inserted through the tube 105 such that its surgery application end 110 is positioned over the in vivo surgical site for tendon repair (FIG. 1B). As will become understood from a review of the illustrations and description that follows, sequenced actuation of various levers of the bioinductive implant placement device 100 will provide for the expansion and placement of the bioinductive implant patch onto the in vivo surgical site.

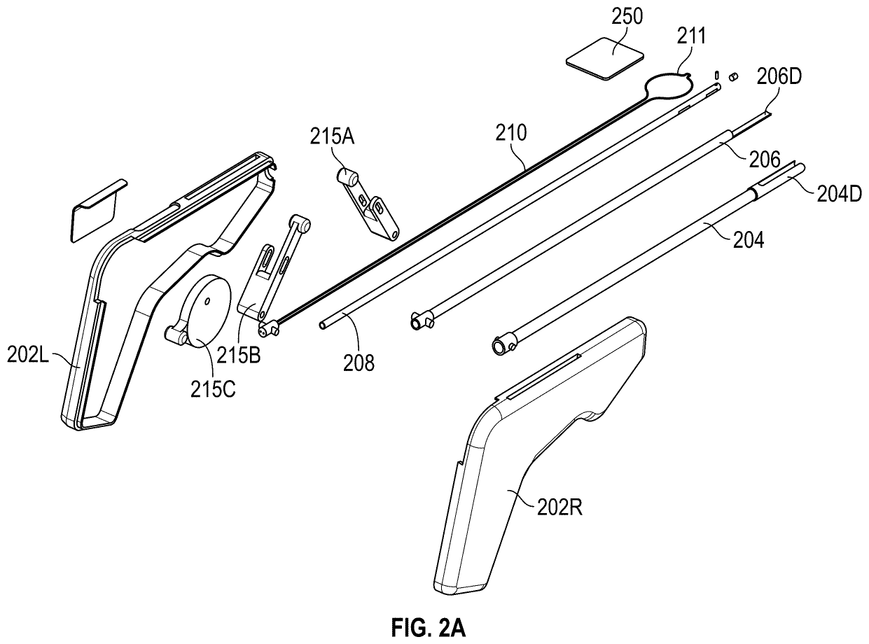

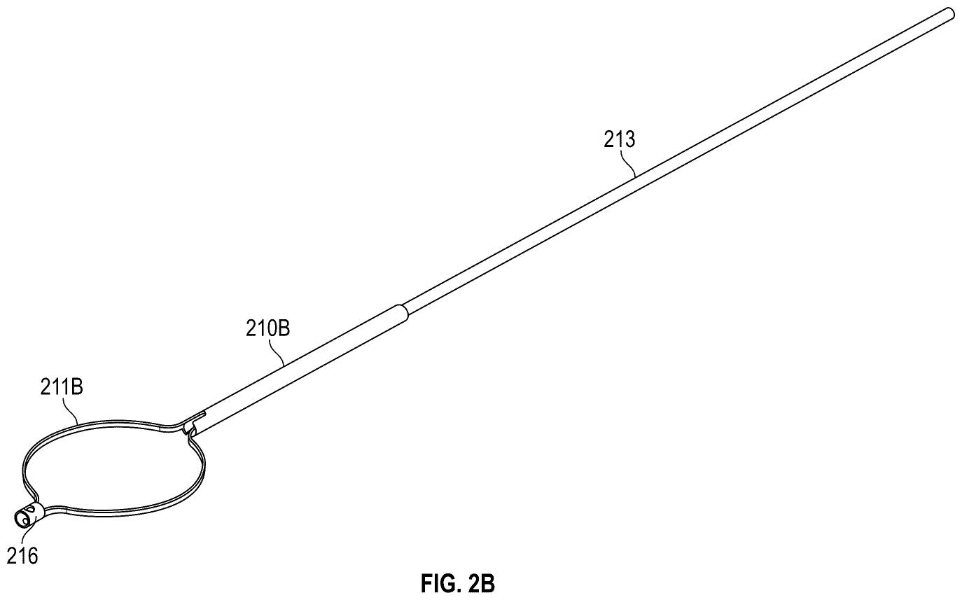

FIG. 2A is an exploded view of an exemplary embodiment of a bioinductive implant placement device 100 according to the solution. A left-side panel 202L and a right-side panel 202R clamshell to form a housing 202 ergonomically shaped to accommodate a user's hand. A tubular sheath assembly 204 extends from the housing 202 and concentrically contains a bottom tab assembly 206 which, in turn, concentrically contains a main shaft 208 which, in turn, supports or carries a patch-splayer component 210A in the form of a Nitinol wire (or laser cut Nitinol tube) with an application head 211A (construction of the patch-splayer component 210A from a Nitinol wire is exemplary-an alternative, exemplary embodiment of a patch-splayer component 210B is shown in FIG. 2B constructed from a laser cut and shape-set Nitinol tube welded to a stainless tube). That is, the main shaft 208 and patch-splayer component 210A assembly is slidably nested within the bottom tab assembly 206 which is slidably nested within the tubular sheath assembly 204. Notably, the main shaft 208 is a stationary, fixed datum relative to which the bottom tab assembly 206 and tubular sheath assembly 204 slidably retract or extend.

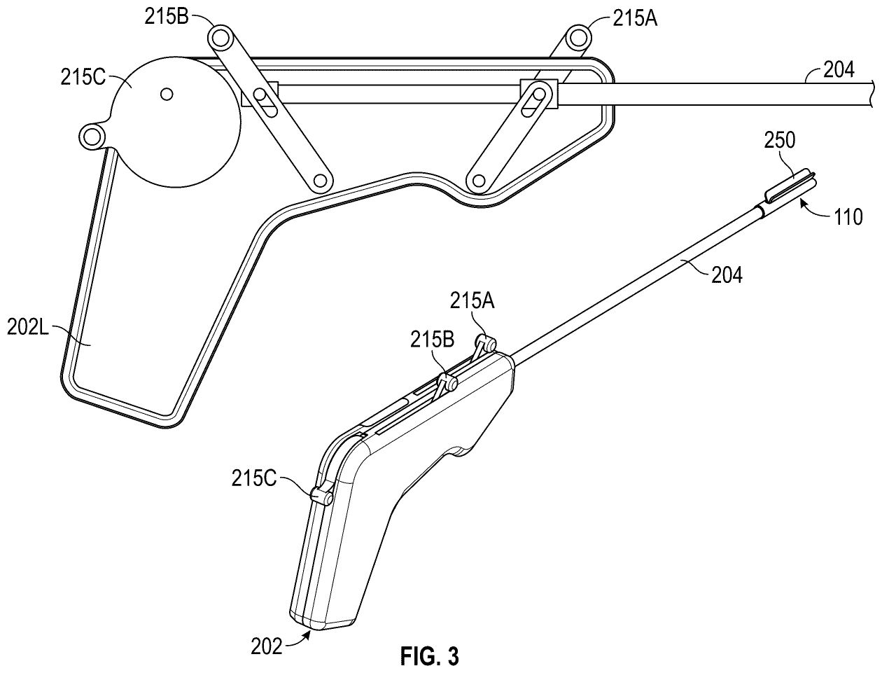

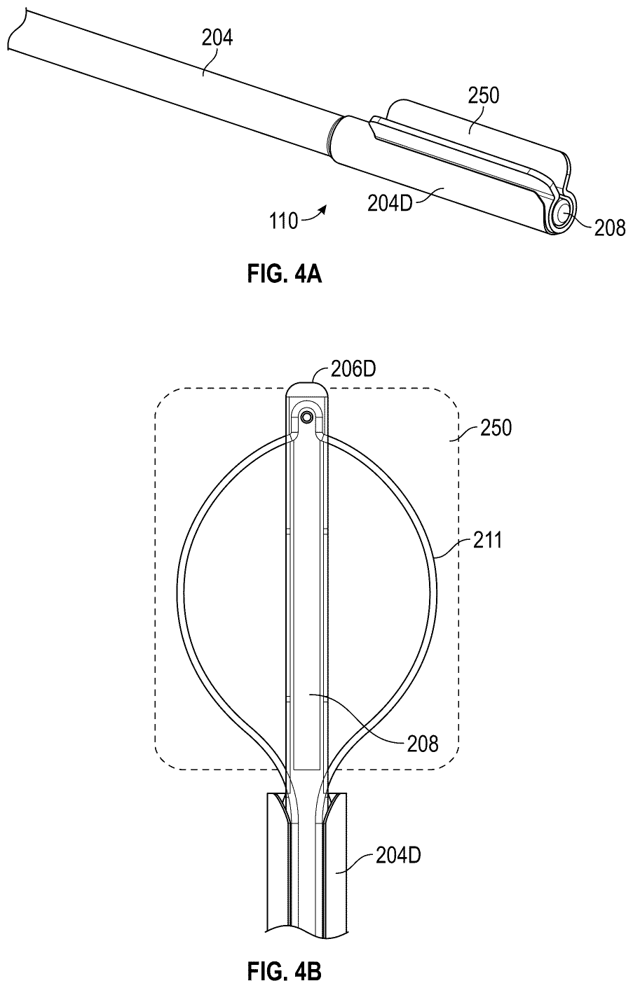

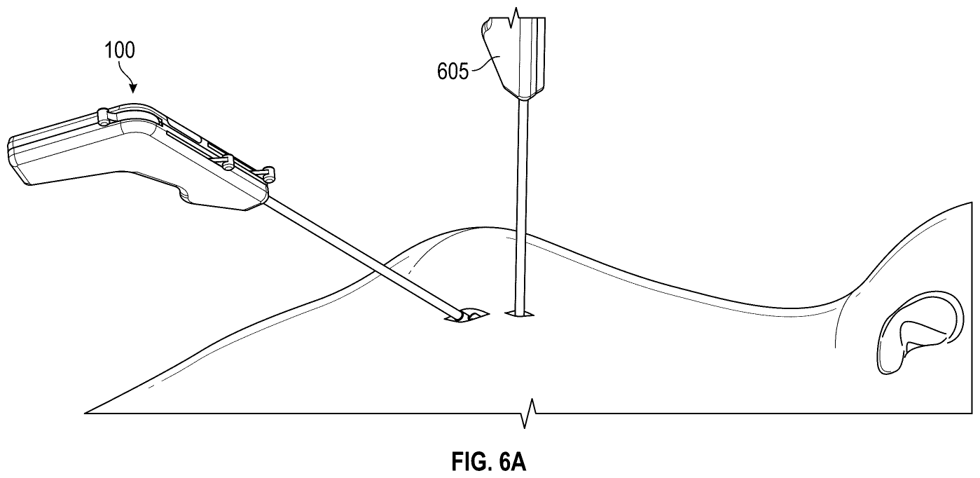

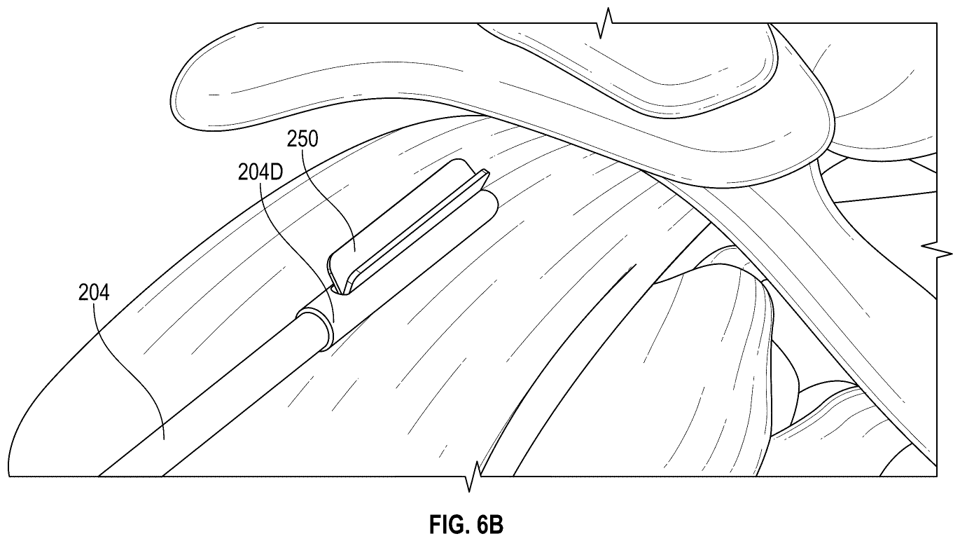

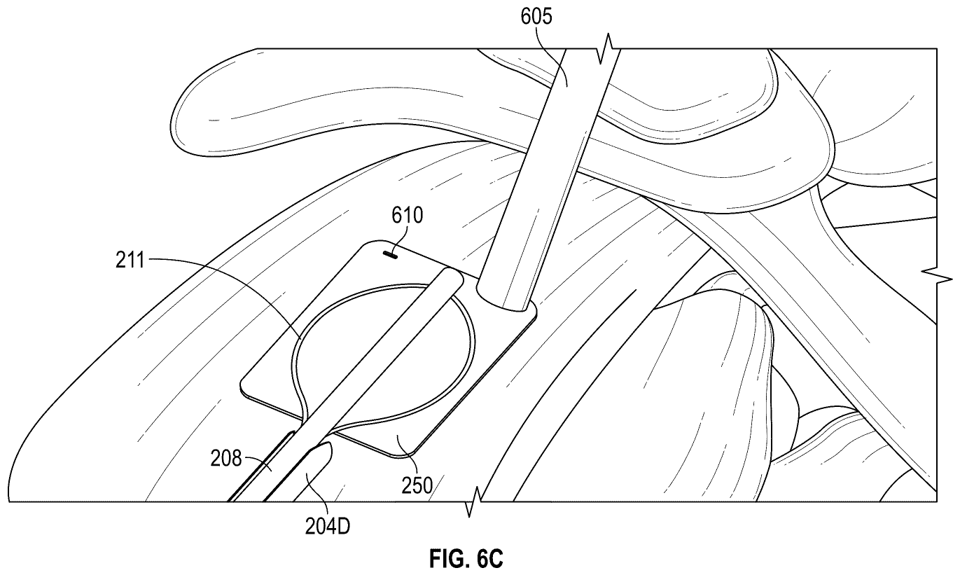

Notably, although Nitinol is the preferred material of the patch-splayer component 210, other materials that are elastically deformable and/or capable of being shape-set and suitable for use in an in vivo surgical environment are envisioned and, as such, the scope of the solution is not limited to use of Nitinol. As would be understood by one of ordinary skill in the art of materials, Nitinol is an alloy of titanium and nickel. Moreover, the patch-splayer component 210 comprises an application head 211 that takes a generally round or oval shape or heart-shape, as can be understood from the FIG. 2A-2B illustrations. The application head 211 is defined by use of a flexible and elastically deformable material such as Nitinol that may be “normally” or “defaulted” to the generally circular shape or heart-shape depicted in the figures such that when no tensioning or pulling force is applied to the application head 211 it defaults to the generally circular shape. As will be become clearer from subsequent illustrations and description, when a tensioning force is applied to the application head 211 by retraction of the patch-splayer component 210 into the tubular sheath assembly 204, the two semi-circular halves of the application head 211 may collapse to fit within the tubular sheath assembly 204 (when the bioinductive implant placement device 100 is in a loaded state-see FIG. 3, FIG. 4A, and FIG. 6B illustrations).

Returning to the FIG. 2A illustration, a series of levers (215A, 215B, and 215C) are mechanically associated, respectively, with the tubular sheath 204, the bottom tab assembly 206, and the patch-splayer component 210A. Application and functionality of the series of levers 215 will be described in more detail below. A bioinductive implant patch 250 is associated with the application head 211A of the Nitinol patch-splayer component 210A. Notably, the tubular sheath 204, at its distal end 204D, takes the form of a fork that defines a slot. The bottom tab assembly 206, at its distal end 206D, takes the form of a lower lip or tab. In this way, and as will become more apparent from figures and descriptions that follow, when the bottom tab assembly 206 is retracted into the tubular sheath 204, the distal end 206D of the bottom tab assembly 206 is aligned beneath the slot defined by the forked distal end 204D of the tubular sheath 204 such that a bioinductive patch 205 is folded around the main shaft 208 and extended out of the top slot of the tubular sheath distal end 204D. The bottom tab assembly 206 extends beneath the patch 205 and holds it in place against the main shaft 208. This is the loaded state of the device 100, ready for insertion to an in vivo surgical site.

Notably, when in the loaded state, the application head 211 is in a collapsed state within tubular sheath 204 due to a tensioning force applied to the patch-splayer component 210 which is anchored to the stationary main shaft 208. That is, with the application head 211 retracted into the tubular sheath 204, the distal end 204D of the tubular sheath 204 retains the application head 211 in its collapsed state (and, by extension, the bioinductive patch 250) in the loaded form/shape wrapped around main shaft 208.

FIG. 2B illustrates an exemplary and alternative patch-splayer component 210B from that exemplary patch-splayer component 210A illustrated in FIG. 2A. The exemplary and alternative patch-splayer 210B is comprised of a laser cut and shape-set Nitinol tube welded to a stainless tube 213 and configured to concentrically receive a main shaft 208. As can be understood from the FIG. 2B illustration, the patch-splayer component 210B has been laser cut and shape-set on its distal end to create an application head 211B. At the very distal end of the application head 211B, an anchor point 216 is configured to receive a main shaft 208, as can be understood from prior illustrations.

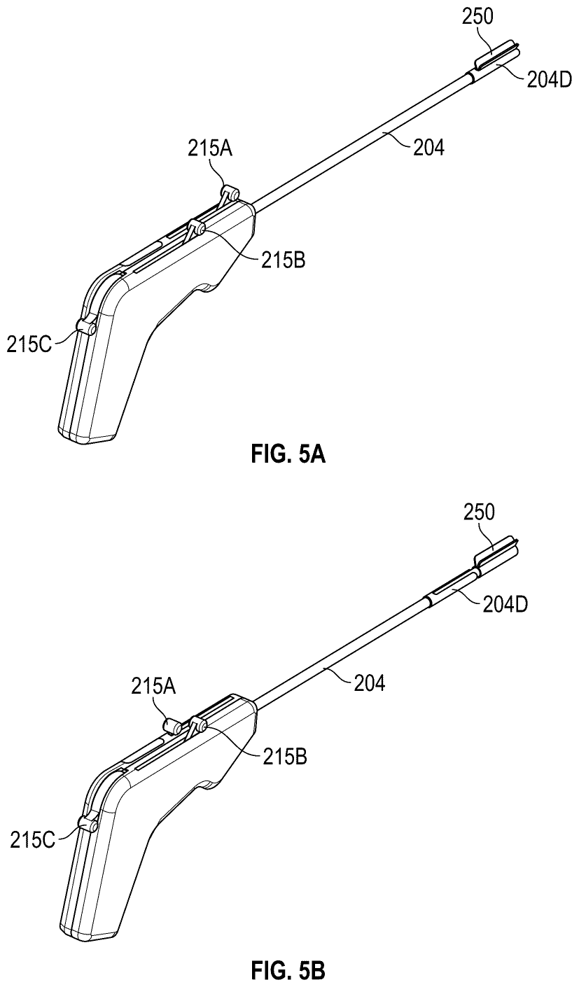

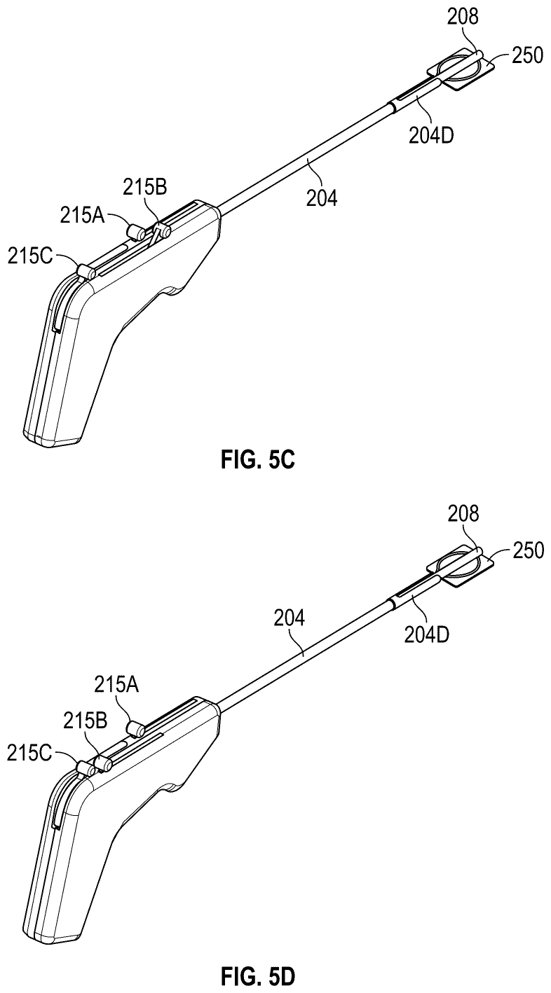

FIG. 3 is a perspective view and cutaway view of the exemplary embodiment of a bioinductive implant placement device 100 shown in FIG. 2. The bioinductive implant placement device 100 is in a loaded state, ready for application of the patch 250 to a surgical site. A close-up view of the distal end 110 of the bioinductive implant placement device 100 when loaded can be seen and understood from the FIG. 4A illustration that follows. In its loaded state, the patch 250 is placed on the application head 211 of the Nitinol patch-splayer component 210 which is “folded” or partially wrapped around the main shaft 208. The bottom tab 206D of the bottom tab assembly 206 is beneath the folded patch/patch-splayer assembly 250/211 to hold the patch 250 in place relative to the main shaft 208. Subsequently, the tubular sheath assembly 204 is extended over the bottom tab assembly 206 (or, depending on perspective, the bottom tab assembly 206 and patch-splayer component 210 are retracted into the tubular sheath 204) such that the forked and slotted distal end 204D aligns with the bottom tab distal end 206D of the bottom tab assembly 206, thereby surrounding with the patch 250 the distal end of the main shaft 208. The application head 211 is collapsed from its expanded state into its retracted state contained within the walls of the tubular sheath 204. The bioinductive implant placement device 100 is loaded and ready for use (FIG. 3, FIG. 4A, and FIG. 5A).

Turning to FIG. 4B, illustrated is a close-up view of the loaded end 110 of the exemplary embodiment of the solution for a bioinductive implant placement device 100 in a deployed state, with the application head 211 of the patch-splayer 210 expanded to splay the bioinductive implant patch 250 from its folded state. Notably, in the FIG. 4B illustration, the bioinductive patch 250 is illustrated as “see-through” in order for the reader to better understand the positioning of the various components of the device 100 relative to each other and the patch 250. As can be understood from the FIG. 4B illustration, the application head 211 is anchored to a pin at the distal end of stationary main shaft 208. The patch 250 is splayed by the application head 211 when the application head 211 is extended from the tubular sheath 204 (via retraction of the tubular sheath 204) and thereby allowed to expand to its default or normal state (as shown in the FIG. 4B illustration). The bottom tab 206D of the bottom tab assembly 206 can be seen still positioned beneath the patch 250. As explained elsewhere in this disclosure, once the patch 250 is placed at the in vivo surgical site, and stapled in place, the bottom tab 206D of the bottom tab assembly 206 may be retracted from beneath the patch 250.

Referring now to FIGS. 5A-5D, illustrated is the actuation sequence of levers 215 for application and release of a bioinductive implant patch 250 to an in vivo surgical site, such as a torn tendon in a rotator cuff. As shown in FIG. 5A, the bioinductive implant placement device 100 is in a loaded state, as previously described. When loaded, the distal end of a bioinductive implant placement device 100 may be inserted to a surgical site (see FIGS. 1, 6A, and 6B). Once positioned over the surgical site, the tubular sheath assembly 204 may be retracted (relative to the main shaft 208, patch-splayer 210/application head 211, and bottom tab assembly 206) by actuation of lever 215A toward the hand of the user and away from the in vivo surgical site (as shown in FIG. 5B). With the tubular sheath assembly 204 retracted, the application head 211 of the patch-splayer component 210 may be allowed to expand by actuation of lever 215C toward the in vivo surgical site (away from the user's hand) such that the lower tab assembly 206 and main shaft 208 remain extended and the application head 211 is allowed to splay out to its default form generally defining a circle (as shown in FIG. 5C). Expansion of the application head 211 operates to “unwrap” the implant patch 250 from around the main shaft 208, thereby allowing the implant patch 250 to be splayed over the in vivo surgical site with the distal end of the lower tab assembly 206D still positioned beneath the patch 250. A close-up view of the “unwrapped” and splayed patch 250 after actuation of lever 215C can be seen and understood from the FIG. 4B and FIG. 6C illustrations.

With the application head 211 of the patch-splayer component 210 in its expanded state, and the bioinductive implant patch 250 splayed over the in vivo surgical site, lever 215B may be actuated toward the user (as shown in FIG. 5D) such that the distal end 206D of the bottom tab assembly 206 is retracted from beneath the bioinductive implant patch 250. Advantageously, with the distal end 206D of the bottom tab assembly 206 retracted, the patch 250 is effectively released from the device 100 and ready to be surgically fixed in place such as by stapling (see FIG. 6C illustration). Notably, it is envisioned that the patch 250 may be partially stapled in place before the user retracts the bottom tab assembly 206 from beneath the patch and/or altogether removes the device 100 from the in vivo surgical site.

FIGS. 6A-6C illustrate in more detail the use of the bioinductive implant placement device 100 according to the solution, with FIG. 6A illustrating arthroscopic access to an in vivo surgical site for an exemplary tendon repair in a shoulder, FIG. 6B illustrating an application end 110 of a bioinductive implant placement device 100 according to the solution in a loaded state and positioned over the in vivo surgical site, and FIG. 6C illustrating the application end 110 of the bioinductive implant placement device 100 according to the solution in a deployed state to place a bioinductive patch 250 over and onto the in vivo surgical site.

Beginning with the FIG. 6A illustration, one of ordinary skill in the art of arthroscopic surgery would understand that two incisions provide access to the in vivo surgical site. In the FIG. 6 illustrations, the exemplary in vivo surgical site is within the shoulder (more specifically, the rotator cuff) of a patient to repair a torn or damaged tendon. In the FIG. 6A illustration, the loaded “surgical end” or application end 110 of the exemplary bioinductive implant placement device 100 is inserted through a first incision while the “surgical end” of a surgical stapler 605 is inserted through a complementary incision. Other incision arrangements or strategies are envisioned.

Turning to the FIG. 6B illustration, the application end 110 is shown in vivo in a loaded state and positioned over the surgical site. The patch 250 is folded around the main shaft 208 by virtue of the application head 211 of the patch-splayer component 210 being retracted into the distal end 204D of the sheath assembly 204. The lip of the bottom tab assembly retains the patch 250 against the main shaft 208. Advantageously, sequential actuation of the various levers 215 as described supra will transition the application end 110 from the loaded state shown in the FIG. 6B illustration to the deployed state shown in FIG. 6C.

Turning now to the FIG. 6C illustration, the application end 110 of the bioinductive implant placement device 100 has been transitioned to its deployed state to place the bioinductive patch 250 over and onto the in vivo surgical site. Retraction of the sheath assembly 204 (or extension of the patch-splayer 210 relative to the tubular sheath 204) has allowed the application head 211 to expand to its default state and thereby splay the patch 250 over the in vivo surgical site. With the patch 250 splayed and positioned, a surgical stapler 605 or other surgical fixation device may be employed to apply staples 610 to fix the patch 250 in place over the damaged area of the tendon. The patch 250 may be completely released from the application end 110 and the bioinductive implant placement device 100 subsequently removed from the in vivo surgical site.

A device and method of use for placement of a bioinductive implant patch in an arthoscopic surgical environment has been described using detailed descriptions and illustrations of an embodiment thereof that is provided by way of example and are not intended to limit the scope of the disclosure. The described embodiment comprises different features, not all of which are required in all embodiments of the solution. Some embodiments of the solution utilize only some of the features or possible combinations of the features. Variations of the embodiment of the solution that is described and illustrated will occur to persons of the art. It will be appreciated by persons skilled in the art that a bioinductive implant placement device according to the solution is not limited by what has been particularly shown and described herein above. Rather, the scope of the disclosed solution is defined by the claims that follow.

What is claimed is:

1. A bioinductive implant placement device comprising:
an ergonomic housing containing a plurality of manual actuators, each actuator extending external to the housing for actuation by a user;
a plurality of concentrically and slidably arranged components extending from the housing to define a surgery application end that is distal to the housing, the plurality of concentrically arranged components comprising a main shaft nested within a bottom tab assembly nested within a tubular sheath; and
an application head of a patch-splayer component coupled to a distal end of the main shaft and configured to transition between a retracted state and an expanded state in response to application of, and/or release of, a tensioning force;
wherein the plurality of manual actuators are each uniquely associated with one of the patch-splayer component, bottom tab assembly, and tubular sheath;
wherein sequential actuation of the plurality of manual actuators causes the surgical application end to transition between a loaded state and a deployed state; and
wherein when in the loaded state the application head of the patch-splayer component is in the retracted state and a bioinductive implant patch is folded for insertion into an in vivo surgical site, and when in the deployed state the application head of the patch-splayer component is in the expanded state and the bioinductive implant patch is splayed and releasable from the surgery application end onto the in vivo surgical site.

2. The bioinductive implant placement device of claim 1, wherein the patch-splayer component is comprised of Nitinol.

3. The bioinductive implant placement device of claim 1, wherein the patch-splayer component is comprised of a wire.

4. The bioinductive implant placement device of claim 1, wherein the patch-splayer component is comprised of a laser cut tube.

5. The bioinductive implant placement device of claim 1, wherein the tubular sheath defines a slot.

6. The bioinductive implant placement device of claim 1, wherein the bottom tab assembly defines a tab that extends beneath the bioinductive implant patch when the surgical application end is in the deployed state.

7. The bioinductive implant placement device of claim 1, wherein, when the application end is in the loaded state, the bioinductive implant patch is folded around the main shaft and the application head of the patch-splayer component is in its retracted state.

8. The bioinductive implant placement device of claim 1, wherein, when the application end is in the deployed state, the application head of the patch-splayer component is in its expanded state such that it takes a default shape that operates to unfold the bioinductive implant patch from around the main shaft.

9. The bioinductive implant placement device of claim 8, wherein the default shape of the application head of the patch-splayer component approximates one of a circle, an oval, and a heart-shape.

10. The bioinductive implant placement device of claim 1, wherein the bioinductive implant patch comprises collagen.

11. A method for using a bioinductive implant placement device according to claim 1, the method comprising:
loading the bioinductive implant device into a loaded state by placing a bioinductive implant patch onto the main shaft, applying a tensioning force to the patch-splayer component, and extending the tubular sheath over the main shaft and application head such that the bioinductive implant patch is folded around the main shaft;
inserting the bioinductive implant device into an in vivo surgical site;
actuating a first manual actuator of the plurality of manual actuators to retract the tubular sheath;
actuating a second manual actuator of the plurality of manual actuators to allow the application head of the patch-splayer component to expand to an expanded shape and splay the bioinductive implant patch over the in vivo surgical site;
surgically fixing the bioinductive implant patch in place in the in vivo surgical site;
actuating a third manual actuator of the plurality of manual actuators to retract the bottom tab assembly from beneath the bioinductive implant patch; and
retracting the bioinductive implant device from the in vivo surgical site.
